(19) United States
(12) Patent Application Publication
(10) Pub. No.: US 2025/0195707 A1
(43) Pub. Date: Jun. 19, 2025

(54) STERILISING CONTAINER, CONTROL DEVICE AND CORRESPONDING SYSTEM

(71) Applicant: Aesculap AG, Tuttlingen (DE)

(72) Inventors: Arne Jansen-Troy, Stockach (DE); Matthias Henke, Villingen-Schwenningen (DE); Daniel Kiessling, Villingen-Schwenningen (DE)

(21) Appl. No.: 18/847,336

(22) PCT Filed: Mar. 16, 2023
(86) PCT No.: PCT/EP2023/056815

§ 371 Date: Sep. 16, 2024

(30) Foreign Application Priority Data
Mar. 17, 2022 (DE) 10 2022 106 282.1

(51) Int. Cl. A61L 2/26; A61B 50/00; A61B 50/30; A61L 2/24

(52) U.S. Cl. CPC A61L 2/26; A61B 50/30; A61L 2/24; A61B 2050/0076; A61L 2202/121; A61L 2202/122; A61L 2202/123; A61L 2202/14; A61L 2202/16; A61L 2202/24

(57) ABSTRACT

A sterilizing container for medical purposes, in particular for medical instruments and the like, includes a base, a cover, a closure unit having a closure element and an actuation element, an energy store, and a control unit designed to control the actuation element by actuation energy stored in the energy store in such a way that the actuation element actuates the closure element. The control unit has a receiver designed to receive actuation signals that trigger actuation of the actuation element. The control unit also has a transmitter designed to send status signals containing information relating to a status of the closure unit. The sterilizing container is designed to secure the cover to the base by actuating the closure element by the actuation element. A control device can externally control the control unit. The sterilizing container and the control device can be parts of a system.

### CROSS-REFERENCE TO RELATED APPLICATIONS

This application is the United States national stage entry of International Application No. PCT/EP2023/056815, filed on Mar. 16, 2023, and claims priority to German Application No. 10 2022 106 282.1, filed on Mar. 17, 2022. The contents of International Application No. PCT/EP2023/056815 and German Application No. 10 2022 106 282.1 are incorporated by reference herein in their entireties.

### FIELD

The present disclosure relates to a sterilization container for medical purposes, in particular to a sterilization container whose lid can be fixed to a bottom element via a closure apparatus, in that a control apparatus controls an actuation element via actuation energy stored in an energy storage such that a closure element is actuated. The present disclosure further relates to a control device for externally controlling the control apparatus of the sterilization container and to a system comprising a sterilization container according to the disclosure and a control device according to the disclosure.

### BACKGROUND

A generic sterilization container is known, for example, from DE 36 32 674 A1 and has a bottom element and a lid. In order to be able to fix the lid to the bottom element, the generic sterilization container has a closure apparatus in which an actuation element actuates a closure element when a control apparatus releases corresponding energy stored in an energy storage. According to DE 36 32 674 A1, this release takes place as a result of sensor signals from sensors attached to the sterilization container.

A disadvantage of the generic sterilization container is that a locked state of the sterilization container only indicates sterility inside the sterilization container immediately after proper sterilization in an autoclave. In order to be able to detect subsequent opening and thus possible contamination of an inner region of the sterilization container, a security seal must be attached to the sterilization container in accordance with DE 36 3 674 A1, for example in accordance with EP 2 896 036 B1.

US 2015/0 374 868 A1 discloses a sterilization container that has a lid that is firmly connected to a bottom trough and can be moved relative to the bottom trough via four actuators. The lid can only be disassembled from the bottom trough by separating the lid and the actuators from each other.

### SUMMARY

With this in mind, the object of the present disclosure is to provide a sterilization container that enables simplified or improved monitoring of a sterilization container in an instrument circuit.

This object is solved by a sterilization container, by a control device and by a corresponding system comprising a sterilization container according to the disclosure and a control device according to the disclosure.

The sterilization container according to the disclosure is configured for medical purposes and is suitable in particular for holding medical or surgical instruments. The sterilization container has a bottom element, a lid, a closure apparatus, an energy storage apparatus and a control apparatus.

The bottom element is configured in particular to be trough-shaped.

The closure apparatus comprises a closure element and an actuation element. In particular, the closure element is a latch-shaped component on the bottom element or the lid, which can interact form-fittingly with a corresponding counterpart on the other part of the sterilization container (lid or bottom element). The actuation element is, in particular, an electric motor that can move or actuate the closure element.

In particular, the energy storage is a battery or an accumulator. Alternatively, the energy storage may also be a mechanical energy storage, for example in the form of a tensionable spring.

The control apparatus is configured to be able to control the actuation element via actuation energy stored in the energy storage in such a way that the actuation element actuates the closure element.

The control apparatus has a receiver which is configured to be able to receive actuation signals which cause actuation of the actuation element. In other words, the control apparatus is configured to receive and evaluate actuation signals via the transmitter and to issue corresponding commands for actuating the closure element to the actuation element. Actuation may mean moving the closure element from a closing or locking position to an opening or unlocking position or moving it from the opening or unlocking position to the closing or locking position. The control apparatus has a transmitter which is configured to be able to send or transmit status signals which contain information about a status of the closure apparatus.

According to the disclosure, the control apparatus is configured to be able to control the actuation element via actuation energy stored in the energy storage in such a way that the actuation element actuates the closure element and thereby fixes the lid to the bottom element. In particular, the lid is sealingly fixed to the bottom element so that no gaseous and/or liquid contamination can enter an inner region of the sterilization container.

In other words, according to the disclosure, the sterilization container is configured to fix the lid to the bottom element by actuating the closure element via the actuation element. In other words, according to the disclosure, the sterilization container is configured such that the lid can be connected to the bottom element or the lid can be separated from the bottom element by actuating the closure element via the actuation element. In other words, the sterilization container is configured in particular in such a way that, by actuating the closure element, the lid can be separated or disassembled from the bottom element or can be brought into a state in which the lid can be separated or disassembled from the bottom element without tools, in particular without further disassembly steps. Preferably, the lid can be brought into a state in which the lid can be separated or disassembled from the bottom element without tools, in particular without further disassembly steps, simply by actuating the closure element in such a way that the closure element is moved from the closing or locking position to the opening or unlocking position.

In particular, the control apparatus may be configured to extract the information about the status of the closure apparatus from a history or recording of previous actuations of the closure element. Alternatively or additionally, the closure apparatus may also have a closure sensor that can detect the status.

The transmitter advantageously provides an interface that can be used to simplify or improve the monitoring of the sterilization container. A corresponding safety seal is no longer necessary.

According to an aspect of the disclosure, the receiver and the transmitter of the control apparatus may be integrally configured in the form of a transceiver. By using a transceiver, the sterilization container according to the disclosure can be implemented at low cost.

According to an aspect of the disclosure, a button may be provided on an outer side of the sterilization container, wherein the button is configured to be able to transmit an actuation signal to the receiver as a result of its activation. In particular, activation of the button may cause the closure element to be opened or unlocked by the actuation element. In particular, the button may be configured in such a way that it can only assume two states (pressed or activated and not pressed or not activated). In particular, the closure element is configured in such a way that it is in the closing or locking position when the actuation element does not actuate the closure element. If the button is pressed (i.e. activated), the control apparatus controls the actuation element in particular in such a way that the closure element is moved from the closing or locking position to the opening or unlocking position.

The use of a button is advantageous compared to conventional, manually openable closures on sterilization containers, since the operation of a button is easier than, for example, the operation of an over dead point lock.

According to an aspect of the disclosure, two buttons may be provided on an outer side of the sterilization container, which are configured to be able to transmit actuation signals to the receiver, and the control apparatus may be configured to actuate the actuation element according to the actuation signals of the buttons only as a result of a synchronous or simultaneous and alike activation of the two buttons.

According to an aspect of the disclosure, two handles may be provided on the outer side of the sterilization container, and the two buttons may each be arranged at or on one of the handles in such a way that a person gripping the sterilization container with his/her hands on the handles can activate one of the two buttons in each case when gripping it with his/her hands. In particular, the handles may be arranged at a distance from each other and/or the buttons may be arranged at a distance from each other in such a way that two-handed gripping of the handles and/or two-handed activation of the buttons is forced. In particular, the handles and/or the buttons may be provided on the lid.

If the button are assigned to handles, the handling of the sterilization container can be controlled in an advantageous manner. If the handles are arranged at the lid, jamming of the lid can be prevented or made more difficult when the lid is lifted.

According to an aspect of the disclosure, an optical and/or acoustic display, which is configured to be able to display the status signals of the transmitter in a form that can be perceived by humans, may be provided on the outer side of the sterilization container. In particular, the display may be configured to display dynamic (i.e. temporally fluctuating) sound and/or light signals.

By providing a display, the provision of peripheral readout devices can be dispensed with, thus further simplifying or improving the monitoring of the sterilization container.

According to an aspect of the disclosure, the button/buttons and the display may be integrally configured in the form of a touch-sensitive display or touch-sensitive monitor or touch screen, or several touch-sensitive displays or touch-sensitive monitors or touch screens.

The intuitive operation of the sterilization container may be enabled or improved by providing touch-sensitive displays. In addition, the sterilizability and cleanability of the button may be improved.

According to an aspect of the disclosure, the receiver may be configured to receive actuation signals wirelessly and/or the transmitter may be configured to transmit status signals wirelessly. In principle, the sterilization container may also be configured in such a way that actuation signals and/or status signals can be read in or out via one or more cables. However, if the transmitter and the receiver of the control apparatus are configured for wireless communication, interfaces for cable connectors can be dispensed with, thus advantageously reducing the surface area of the sterilization container that needs to be cleaned.

According to an aspect of the disclosure, the closure apparatus may have an emergency opening element which is configured to be removable only via a tool and to cancel the effect of the closure element when the emergency opening element alone is disassembled. In particular, the closure element may have an undercut and the portion of the closure element at which the undercut is configured may be connected to the remaining part of the closure element via the emergency opening element in the form of a screw or another connection element. Alternatively, a part of the actuation element that can interact with the closure element in an assembled state may also be connected to the remaining part of the actuation element via the emergency opening element in the form of a screw or another connection element. Alternatively, a part of the sterilization container, which in the closed state of the sterilization container interacts form-fittingly with the closure element in its locking position, may also be connected to the remaining part of the sterilization container via the emergency opening element in the form of a screw or another connection element. Preferably, the emergency opening element is configured in such a way that it can only be removed using a key as a tool.

By providing the emergency opening element, access to the inside of the sterilization container can be guaranteed in the event of a failure of the control apparatus.

According to an aspect of the disclosure, the energy storage may be configured to be charged wirelessly. In particular, the energy storage may comprise an induction coil for wireless charging.

If the energy storage is configured in such a way that it can be charged wirelessly, it is advantageously possible to use an energy storage with a low energy storage capacity without having to compromise on functionality.

A control device according to the disclosure is suitable for externally controlling a control apparatus of a sterilization container according to the disclosure. ‘External’ means in particular that the control device is not part of the sterilization container or is not permanently connected to the sterilization container.

The control device according to the disclosure has a transmitter (transmitter of the control device as opposed to the transmitter of the control apparatus) which is configured to be able to transmit actuation signals to the control apparatus, and has a receiver (receiver of the control device as opposed to the receiver of the control apparatus) which is configured to be able to receive status signals from the control apparatus. Preferably, the transmitter and the receiver of the control device are configured integrally in the form of a transceiver. In particular, the control device is configured to be able to communicate wirelessly with the control apparatus of the sterilization container. In other words, the control device is configured in particular as a remote control for the sterilization container. In particular, the external control device is configured on or in an autoclave.

If an external control device is provided, the degree of automation of a sterilization process can be increased.

According to an aspect of the disclosure, the control device may comprise a temperature sensor, a pressure sensor and/or a humidity sensor and may be configured to emit an actuation signal, in particular to the control apparatus of the sterilization container, when a predetermined temperature, a predetermined pressure and/or a predetermined humidity is measured or when reaching a predetermined number of repeated measurements of the predetermined temperature, of the predetermined pressure and/or of the predetermined humidity.

If the sensors are arranged in a remote control, the manufacturing costs for the sterilization container can be reduced.

A system according to the disclosure comprises a sterilization container according to the disclosure and a control device according to the disclosure. The control apparatus of the sterilization container of the system according to the disclosure is configured to be able to determine whether the control device is located in a predetermined region around the sterilization container. Furthermore, the control apparatus is configured to only be able to transmit an actuation signal to the actuating apparatus as long as the control device is located in the predetermined region around the sterilization container. The determination of whether the control device is located in a predetermined region around the sterilization container can be made on the basis of an evaluation, running in the control apparatus, of signals that the receiver of the control apparatus receives from the transmitter of the control device. In particular, the determination may be based on the signal strength of these signals.

If actuation of the actuation element is only permitted when the sterilization container is in the vicinity of the control device, i.e. in a predetermined region around the control device, a proper course of the sterilization can be facilitated.

### BRIEF DESCRIPTION OF THE DRAWINGS

The present invention is described in more detail below with reference to the accompanying drawings via a preferred configuration example. The following is shown:

FIG. 1 shows a side view of a sterilization container according to the disclosure; and

FIG. 2 shows a cross-sectional view of the sterilization container according to the disclosure corresponding to line II-II of FIG. 1.

### DETAILED DESCRIPTION

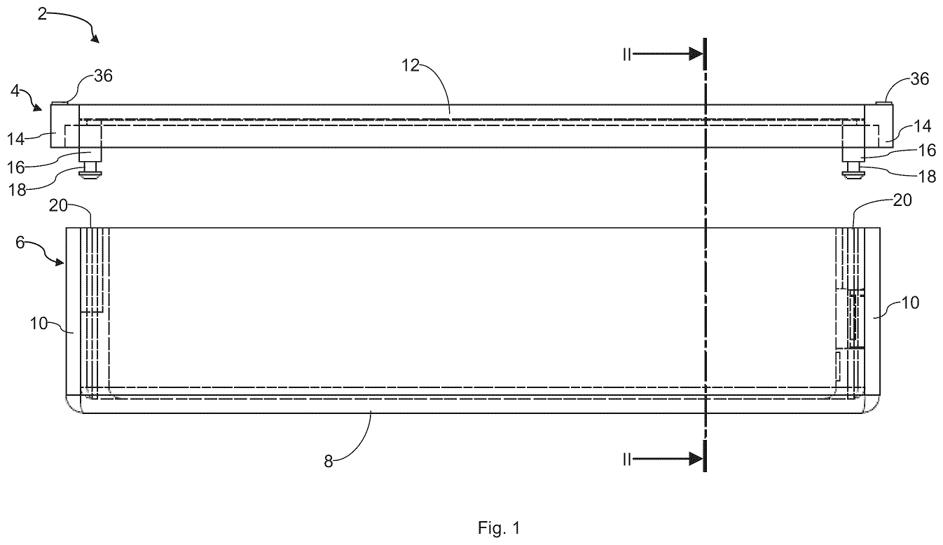

FIG. 1 shows a side view of a sterilization container 2 according to the disclosure with a lid 4 and a bottom element 6. The bottom element 6 is trough-shaped and has a bottom 8 with a substantially rectangular outline and four substantially rectangular sidewalls 10 extending from the four edges of the bottom 8.

The lid 4 has a cover part 12 with a substantially rectangular outline and four substantially rectangular rims 14 extending from the four edges of the cover part 12.

The lid 4 and the rims 14 are dimensioned in such a way that free edges of the sidewalls 10 are in contact with the cover part 12 and/or the rims 14 of the lid 4 via a seal (not shown) when the lid 4 is placed on the bottom element 6 in a closed state of the sterilization container 2. In the closed state of the sterilization container 2, the rims 14 overlap the free edges of the sidewalls 10. The seal of the sterilization container is designed in such a way that in the closed state of the sterilization container 2, liquid and/or gaseous contaminants cannot penetrate into an inner region defined by the bottom 8, the cover part 12, the sidewalls 10 and the rims 14.

In order to fix the lid 4 in the closed state of the sterilization container 2, pins 16 are provided on a side of the cover part 12 facing the inner region. The pins 16 extend from an area of the cover part 12 inside the rims 14, on which the free edges of the sidewalls rest on the cover part 12 via the seal (not shown) in the closed state of the sterilization container 2. An undercut in the form of a circumferential groove 18 is provided at each of the free ends of the pins 16, so that the free ends of the pins 16 are mushroom-shaped.

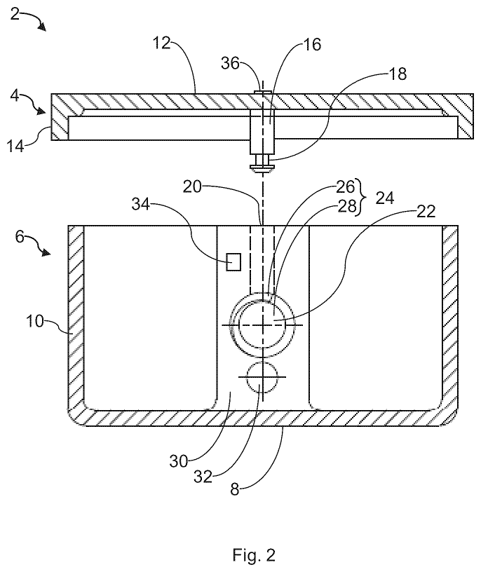

The lid 4 has two pins 16, each of which is arranged centrally on one of the short rims 14 (see also FIG. 2).

Corresponding to the pins 16, a pin receptacle 20 is provided on each of the free rims of the sidewalls 10, more precisely on the short sidewalls 10, in which the pins 16 are received in the closed state of the sterilization container 2.

The interaction of the pins 16 with the bottom element 6 is explained below only with regard to one pin 16. The sterilization container 2 is configured symmetrically in such a way that these explanations also apply to the other pin 16.

FIG. 2 shows a cross-sectional view of the sterilization container 2 corresponding to line II-II of FIG. 1.

As shown in FIG. 2, a closure element 22 in the form of an eccentric is provided at an end of the pin receptacle 20 facing away from the corresponding free rim of the sidewall 10, which can be moved or actuated via an actuator (not shown) (for example an electric motor). The actuator thus represents an actuation element according to the disclosure. The actuator can be operated with the aid of an energy storage device (not shown).

The closure element 22 has a cam disk 24, the cam 26 of which is arranged on an axial side or end face of a disk 28 with a round contour. The cam 26 extends along an area (approximately a quarter) of the circumference of the disk 28 and has a wedge-shaped outline running along the circumference of the disk 28. The closure element 22 is arranged in the corresponding sidewall 10 at the pin receptacle 20 in such a way that the disk 28 extends parallel to the sidewall 10 and the cam 26 can be moved into or out of the pin receptacle 20 as desired.

When the pin 16 is received in the pin receptacle 20 in the closed state of the sterilization container 2, the closure element 22 can be moved or rotated via the actuator in such a way that the cam 26 engages in the circumferential groove 18 of the pin 16 in a locking position (see FIG. 2) and thus prevents a relative movement of the pin 16 out of the pin receptacle 20. In order to release the pin 16, the closure element 22 can be rotated in such a way that the cam 26 does not engage in the pin receptacle in an opening position and therefore cannot interact form-fittingly with the circumferential groove 18 of the pin 16.

Due to the wedge-shaped design of the cam 26, actuation or movement or rotation of the closure element 22 can cause a relative movement of the pin 16 along the direction in which the pin receptacle 20 extends when the pin 16 is held in the pin receptacle 20. This makes it possible to lift the lid 4 with the aid of the closure element 22 or to put less stress on the seal (not shown) and to transfer the sterilization container 2 from the closed state to an open or unsealed state.

In order to be able to control the actuator integrated in the corresponding sidewall 10 (not shown), a control apparatus 30 is also integrated in the corresponding sidewall 10. The control apparatus 30 has a sensor 32 which can measure a pressure, a temperature and/or a humidity in the inner region of the sterilization container 2.

According to the disclosure, the control apparatus 30 is equipped with a transceiver 34. On an outer side of the lid 4, buttons 36 are provided at rim regions (see also FIG. 1). The buttons 36 are configured in such a way that they can emit an actuation signal when they are pressed. The control apparatus 30 is configured in such a way that it actuates the actuator (not shown) when the transceiver 34 receives matching actuation signals from both buttons 36. The control apparatus 30 is configured to transmit via the transceiver 34 the status of the closure element 22 or the status of the closure apparatus formed by the pin 16, the pin receptacle 20, the closure element 22 and the actuator (not shown) via status signals.

According to the disclosure, the control apparatus 30 is also configured to receive actuation signals for actuating the closure element 22 via the transceiver 34 not only from the button 36, but also from an external control device (not shown).

### LIST OF REFERENCE SIGNS

2 sterilization container
4 lid
6 bottom element
8 bottom
10 sidewall
12 cover part
14 rim of the lid
16 pin
18 circumferential groove
20 pin receptacle
22 closure element
24 cam disk
26 cam
28 disk
30 control apparatus
32 sensor
34 transceiver
36 button

1.-13. (canceled)

14. A sterilization container for medical purposes, the sterilization container comprising:
a bottom element;
a lid;
a closure apparatus comprising a closure element and an actuation element;
an energy storage; and
a control apparatus,
the control apparatus being configured to control the actuation element via actuation energy stored in the energy storage such that the actuation element actuates the closure element,
the control apparatus comprising a receiver configured to receive at least one actuation signal that causes actuation of the actuation element,
the control apparatus also comprising a transmitter configured to transmit a status signal that contains information about a status of the closure apparatus,
the sterilization container being configured to fix the lid to the bottom element by actuating the closure element via the actuation element, and
the sterilization container being configured such that actuation of the closure element permits the lid to be disassembled from the bottom element without tools.

15. The sterilization container according to claim 14, wherein the receiver and the transmitter are integrally configured in the form of a transceiver.

16. The sterilization container according to claim 14, further comprising at least one button on an outer side of the sterilization container, wherein the at least one button is configured to send the at least one actuation signal to the receiver when the at least one button is activated.

17. The sterilization container according to claim 16, wherein:
the outer side comprises a first outer side portion and a second outer side portion,
the at least one button comprises a first button on the first outer side portion and a second button on the second outer side portion,
the at least one actuation signal comprises a first actuation signal and a second actuation signal,
the first button and the second button are configured to transmit the first actuation signal and the second actuation signal, respectively, to the receiver, and
the control apparatus is configured to actuate the actuation element according to the first actuation signal of the first button and the second actuation signal of the second button only as a result of a simultaneous activation of the first button and the second button.

18. The sterilization container according to claim 17, further comprising a first handle on the first outer side portion and a second handle on the second outer side portion, wherein the first button is arranged on the first handle, and the second button is arranged on the second handle, such that each of the first button and the second button is activatable by a person gripping the sterilization container with two hands.

19. The sterilization container according to claim 16, further comprising an optical and/or acoustic display on the outer side of the sterilization container, the optical and/or acoustic display configured to display status signals of the transmitter, wherein the at least one button and the optical and/or acoustic display are integrally configured as a touch-sensitive display or several touch-sensitive displays.

20. The sterilization container according to claim 14, further comprising an optical and/or acoustic display on an outer side of the sterilization container, the optical and/or acoustic display configured to display the status signal of the transmitter.

21. The sterilization container according to claim 14, wherein the receiver is configured to receive the at least one actuation signal wirelessly and/or the transmitter is configured to transmit the status signal wirelessly.

22. The sterilization container according to claim 14, wherein the closure apparatus has an emergency opening element configured to be removable only via a tool and to cancel an effect of the closure element when the emergency opening element alone is disassembled.

23. The sterilization container according to claim 14, wherein the energy storage is configured to be charged wirelessly.

24. A system comprising:
a sterilization container according to claim 14; and
a control device for externally controlling the control apparatus,
wherein the control device comprises a control transmitter configured to transmit the at least one actuation signal to the control apparatus, and a control receiver configured to receive the status signal from the control apparatus.

25. The system according to claim 24, wherein:
the control device comprises a temperature sensor and/or a pressure sensor and/or a humidity sensor,
the control device being configured to emit the at least one actuation signal when a predetermined temperature, a predetermined pressure and/or a predetermined humidity is measured or when reaching a predetermined number of repeated measurements of the predetermined temperature, of the predetermined pressure and/or of the predetermined humidity.

26. The system according to claim 24, wherein the control apparatus is configured to determine whether the control device is located in a predetermined region around the sterilization container, and to transmit the at least one actuation signal to an actuating apparatus only as long as the control device is located in the predetermined region around the sterilization container.
